(19) United States
(12) Patent Application Publication
(10) Pub. No.: US 2016/0015011 A1
(43) Pub. Date: Jan. 21, 2016

(54) ANIMAL MODEL RESISTANT TO HEARING LOSS

(71) Applicant: Tohoku University, Miyagi (JP)

(72) Inventors: Jun Suzuki, Miyagi (JP); Noriko OSUMI, Miyagi (JP); Yusuke TAKADA, Miyagi (JP); Yuji OWADA, Yamaguchi (JP)

(21) Appl. No.: 14/426,202

(22) PCT Filed: Sep. 13, 2013
(86) PCT No.: PCT/JP2013/074940

§ 371 Date: Oct. 6, 2015

(30) Foreign Application Priority Data
Sep. 13, 2012 (JP) 2012-202027

(51) Int. Cl. A01K 67/027; G01N 30/72; G01N 27/447

(52) U.S. Cl. CPC A01K 67/0276; G01N 27/44717; G01N 30/72; A01K 2207/05; A01K 2227/105; A01K 2267/035

(57) ABSTRACT

The present invention provides an animal model resistant to hearing loss by producing a fatty acid binding protein 7 gene knockout animal.

### CROSS REFERENCE TO RELATED APPLICATIONS

The present application claims the benefit of Japanese Patent Application No. 2012-202027 filed on Sep. 13, 2012, the entire disclosure of which is hereby incorporated by reference.

### TECHNICAL FIELD

The present invention relates to animal models resistant to hearing loss.

### BACKGROUND ART

Fatty acid binding protein 7 (Fabp7) is one of fatty acid binding proteins that bind unsaturated fatty acids such as DHA and are involved in intracellular transport of fatty acids (Nature reviews Drug discovery. 2008; 7(6): 489-503). Fabp7 is highly expressed in the brain and is thought to be involved in the proliferation and maintenance of neural stem cells (Stem Cells. 2012; 30(7): 1532-43). In addition, in the cochlear of the inner ear, the expression of Fabp7 has been detected in satellite cells in the spiral ganglion, supporting cells in the organ of Corti, and fibrocytes in the spiral limbus or spiral ligament. Function of Fabp7 in these tissues, however, remains unknown (Annals of anatomy. 2010; 192(4): 210-4).

On the other hand, in genetic studies on age-related hearing loss in the past, there have been many reports showing that the absence of a certain gene results in congenital hearing loss or a phenotype of accelerated progression of age-related hearing loss. In contrast, very few reports showed delay of onset or progression of age-related hearing loss in knockout mice in which a certain gene is deleted, including the one showing that the onset of age-related hearing loss can be significantly prevented in knockout mice deficient in a gene encoding Bak, a kind of apoptosis-promoting proteins (Proc Natl Acad Sci USA. 2009 Nov. 17; 106(46): 19432-7).

### Problem to be Solved by the Invention

An object of the present invention is to provide animal models resistant to hearing loss.

### SUMMARY OF THE INVENTION

The inventors have found, during analyses of phenotypes of Fabp7 knockout mice, that the onset and progression of age-related hearing loss are delayed in these mice. Histological analyses showed that degeneration of the cochlea with aging was kept moderate and the loss of neurons, fibrocytes, and hair cells was suppressed. In addition, these knockout mice were resistant to acoustic exposure and showed reduction of temporary elevation of hearing threshold after acoustic exposure which would occurs in wild-type mice. The inventors thus accomplished the following inventions.

An aspect of the present invention is an animal model resistant to hearing loss being a fatty acid binding protein 7 gene knock-out mouse. The hearing loss may be age-related in the animal model, in which onset or progression of the age-related hearing loss is delayed. Alternatively, the hearing loss may be noise-induced in the animal model, in which the noise-induced hearing loss is suppressed.

Another aspect of the present invention is a method of measuring hearing with the passage of age in the animal model. A further aspect is a method of measuring hearing after acoustic exposure in the animal model.

A yet another aspect of the present invention is a method of screening for a gene involved in age-related hearing loss, the method comprising searching for a gene showing expression in the animal model in a manner different from that of the gene in an animal exhibiting normal age-related hearing loss. A still another aspect is a method of screening for a gene involved in noise-induced hearing loss, the method comprising searching for a gene showing expression in the animal model in a manner different from that of the gene in an animal exhibiting normal hearing loss due to acoustic exposure. In these methods, the searching may be performed on the basis of expression at a transcriptional level or expression at a protein level.

Another aspect of the present invention is method of screening for a substance being a marker for age-related hearing loss, the method comprising searching for a compound present in the animal model in an amount or a concentration different from that of the compound in an animal exhibiting normal age-related hearing loss. A yet another aspect is a method of screening for a substance as a marker for noise-induced hearing loss, the method comprising searching for a compound present in the animal model in an amount or a concentration different from that of the compound in an animal exhibiting normal noise-induced hearing loss.

A further aspect of the present invention is a method of producing an animal model with delayed onset and/or progression of age-related hearing loss, the method comprising suppressing expression of fatty acid binding protein 7 gene in an animal. A still further aspect is a method of producing an animal model with reduced noise-induced hearing loss, the method comprising suppressing expression of fatty acid binding protein 7 gene in an animal.

A yet further aspect of the present invention is a method of delaying onset or progression of age-related hearing loss in a non-human vertebrate, the method comprising suppressing expression of fatty acid binding protein 7 gene in the vertebrate. A still further aspect is a method of reducing noise-induced hearing loss in a non-human vertebrate, the method comprising suppressing expression of fatty acid binding protein 7 gene in the vertebrate.

Another aspect of the present invention is a method of screening for a compound capable of delaying onset or progression of age-related hearing loss, the method comprising examining whether a candidate compound inhibits expression of fatty acid binding protein 7. A further aspect is a method of screening for a compound capable of reducing noise-induced hearing loss, the method comprising examining whether a candidate compound inhibits expression of fatty acid binding protein 7.

A yet further aspect of the present invention is a marker for age-related hearing loss or noise-induced hearing loss, the marker being a fatty acid binding protein 7 gene.

### BRIEF DESCRIPTION OF THE DRAWINGS

FIG. 1 represents a figure showing results obtained by examining expression of Fabp7 protein in the cochlea of (A) a young wild-type mouse (Fabp7 (+/+)) at 2 months of age, (B) a Fabp7 heterozygous knockout mouse (Fabp7 (+/−)), and (C) a Fabp7 knockout mouse (Fabp7 (−/−)), in an example of the present invention.

FIG. 2 represents a figure showing results obtained by examining expression of Fabp7 protein in the cochlea of (A) a old wild-type mouse (Fabp7 (+/+)) at 12 months of age and (B) a Fabp7 knockout mouse (Fabp7 (−/−)), in an example of the present invention.

FIG. 3 represents a figure showing results of histological analysis of the cochlea of (A to G) old wild-type mice (Fabp7 (+/+)) and (H to N) Fabp7 knockout mice (Fabp7 (−/−)), all at 12 months of age, and a histogram showing loss of cells in Fabp7 (+/+) and Fabp7 (−/−) mice (0 to P) at 12 months of age and (Q to R) at 15-20 months of age, in an example of the present invention.

FIG. 4 represents a figure showing results obtained by analysis of the number of outer hair cells (OHCS) in organ of Corti in apical turn of (A) a wild-type mouse (Fabp7 (+/+)) and (B) a Fabp7 knockout mouse (Fabp7 (−/−)), both at 12 months of age, and represents (C) a histogram showing loss of cells (%) in the Fabp7 (+/+) and Fabp7 (−/−) mice at 7 and 12 months of age, in an example of the present invention.

FIG. 5 represents a figure showing delayed onset and/or progression of age-related hearing loss in Fabp7 knockout mice by measuring hearing thresholds using auditory brain-stem response (ABR) in wild-type mice (Fabp7 (+/+)), Fabp7 heterozygous knockout mice (Fabp7 (+/−)), and Fabp7 knockout mice (Fabp7 (−/−)) at (A) 7 months of age, (B) 12 months of age, and (C) 15-20 months of age, in an example of the present invention.

FIG. 6 represents a figure showing results of experiments in which wild-type mice (Fabp7 (+/+)), Fabp7 heterozygous knockout mice (Fabp7 (+/−)), and Fabp7 knockout mice (Fabp7 (−/−)), all at 2 months of age, were exposed to noise to induce temporary hearing loss and then hearing thresholds were measured using auditory brain-stem response (ABR). FIG. 6A shows a summary of the experiments (Note: alphabetical letters following ABR indicate the corresponding audiograms in this figure), FIG. 6B shows hearing thresholds before acoustic exposure in temporary threshold shift (TTS) experiments, FIG. 6C shows a shift in hearing threshold 4 hours after acoustic exposure in the TTS experiments, and FIG. 6D shows a shift in hearing threshold 7 days after acoustic exposure in the TTS experiments, in an example of the present invention.

### EMBODIMENTS FOR CARRYING OUT THE INVENTION

Unless otherwise noted in embodiments and examples, all procedures used are according to standard protocols such as J. Sambrook, E. F. Fritsch & T. Maniatis (Ed.), Molecular cloning, a laboratory manual (3rd edition), Cold Spring Harbor Press, Cold Spring Harbor, New York (2001); and F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, commercial reagent kits or measurement instruments are used as described in protocols attached thereto, unless otherwise noted.

The above and further objects, features, advantages, and ideas of the present invention are apparent to those skilled in the art from the description of this specification. Furthermore, those skilled in the art can easily reproduce the present invention from the description herein. The embodiments and specific examples described below represent preferable embodiments of the present invention, which are given for the purpose of illustration or explanation. The present invention is not limited thereto. It is obvious to those skilled in the art that various changes and modifications may be made according to the description of the present specification within the spirit and scope of the present invention disclosed herein.

==Animal Models with Delayed Onset and/or Progression of Age-Related Hearing Loss==

The animal model resistant to hearing loss in the present invention is fatty acid binding protein 7 (Fabp7) gene knockout mice. Cause of the hearing loss is not specifically limited and the hearing loss may be age-related hearing loss due to aging, or temporary, long-term, or permanent hearing loss due to exposure to noise (which are collectively referred to as noise-induced hearing loss herein). The hearing loss means significant reduction in hearing for a sound at a certain frequency.

Since these animals exhibit a phenotype that onset and/or progression of age-related hearing loss is delayed, they can be used as an animal model with delayed onset and/or progression of age-related hearing loss. The age-related hearing loss corresponding to presbycusis in human refers to bilateral sensorineural hearing loss which progresses with age.

In addition, since another phenotype of reduction of temporary elevation of hearing threshold due to acoustic exposure is observed, the animals can be used as an animal model with reduced noise-induced hearing loss.

### <Production Method>

The animal models can be produced by suppressing the expression of Fabp7 protein in animals.

The species of the animals is not specifically limited as long as they have the cochlear of the inner ear. Examples include vertebrates represented by laboratory animals such as mice, rats, marmosets, and monkeys.

The method of suppressing the expression of Fabp7 protein is not specifically limited and examples include a knockout technique which disrupts an endogenous gene or a knockdown technique using, for example, siRNA. It is preferable that the expression is almost completely suppressed in any methods.

The tissue where the expression of Fabp7 protein is suppressed may be the cochlear of the inner ear alone, but the expression may be suppressed in the entire body.

### <Method of Use>

It is expected to elucidate a mechanism of onset of hearing loss such as age-related hearing loss and noise-induced hearing loss by analyzing the cochlea which is the auditory organ of the inner ear using these animal models. For example, it is possible to reveal a gene network responsible for hearing loss, in particular, a gene network involved in onset and/or progression of hearing loss such as age-related hearing loss and noise-induced hearing loss, by searching for candidate genes showing expression in these animal models in a manner different from that of the genes in the animals exhibiting normal age-related hearing loss or noise-induced hearing loss using a microarray technique and then actually examining their expressions in detail at the transcriptional or protein level to identify genes downstream of Fabp7.

In addition, by metabolomic analysis in these animal models it is possible to screen for substances (in particular, lipid metabolites) that can serve as markers for hearing loss such as age-related hearing loss and noise-induced hearing loss. For example, it is possible to identify markers for hearing loss such as age-related hearing loss and noise-induced hearing loss by searching for compounds present in these animal models in an amount or a concentration different from that of the compounds in the animals exhibiting normal age-related hearing loss, using, for example, CE/MS (capillary electrophoresis-mass spectrometry), LC/MS (liquid chromatography-mass spectrometry), or GC/MS (gas chromatography-mass spectrometry), and then examining the selected compounds for correlations between, for example, their serum level and hearing loss such as age-related hearing loss and noise-induced hearing loss. Since the compounds thus obtained can serve as candidate substances capable of promoting or delaying onset or progression of hearing loss such as age-related hearing loss and noise-induced hearing loss, they may be screened by examining whether they can promote or delay the onset or progression of the hearing loss such as age-related hearing loss and noise-induced hearing loss.

Thus, if a mechanism of onset of hearing loss such as age-related hearing loss and noise-induced hearing loss can be elucidated, it is further expected that agents for preventing onset and/or delaying progression of hearing loss such as age-related hearing loss and noise-induced hearing loss can be developed based on this mechanism.

### ==Method of Delaying Age-Related Hearing Loss==

The Fabp7 gene knockout animals exhibit phenotypes of being resistant to hearing loss, in particular, a phenotype of delay of onset and progression of age-related hearing loss as well as reduction of temporary elevation of hearing threshold due to acoustic exposure. Accordingly, in human or non-human animals, it is possible to make them resistant to hearing loss, in particular, to delay onset and/or progression of age-related hearing loss or reduce a temporary elevation of hearing threshold due to acoustic exposure, by suppressing expression or function of Fabp7 protein. The method used to suppress the expression of fatty acid binding protein 7 protein is not specifically limited and examples include a knockdown technique using, for example, siRNA. In order to suppress its function, an inhibitory antibody or a low-molecular compound which binds to an active site of Fabp7 protein can be considered.

Furthermore, expression suppressors that suppress the expression of fatty acid binding protein 7 protein as well as function suppressors that suppress its function may be used as agents for preventing onset and/or delaying progression of hearing loss such as age-related hearing loss and noise-induced hearing loss. Specific examples include antisense nucleic acids, siRNA, miRNA, shRNA, aptamers, and antibodies inhibiting its function. It is possible to prevent onset of hearing loss such as age-related hearing loss and noise-induced hearing loss by administering the agent(s) before the onset of the hearing loss such as age-related hearing loss and noise-induced hearing loss. Even if hearing loss such as age-related hearing loss and noise-induced hearing loss has been developed, its progression can be delayed by administering the agent(s) after that.

The way of administration is varied depending on active ingredients of the preventing or delaying agent. If the agent is a nucleic acid drug, direct routes of administration such as injection are preferable. If it is a low-molecular compound, indirect routes of administration such as oral administration are preferable. In any case, a person in charge of administration can choose an appropriate way of administration.

==Method of Screening for Compound Capable of Preventing Onset and/or Delaying Progression of Hearing Loss==

The agents for preventing onset and/or delaying progression of hearing loss such as age-related hearing loss and noise-induced hearing loss as described above can be obtained by screening for compounds capable of inhibiting function or the expression of Fabp7. It can be determined whether a candidate compound inhibits function or expression of Fabp7 by adding the candidate compound to cultured cells expressing Fabp7 and examining reduction of the expression of Fabp7. The compound may be either a low-molecular compound or a high-molecular compound such as nucleic acid.

### ==Markers for Age-Related Hearing Loss==

The marker for age-related hearing loss according to the present invention is Fabp7 gene. More specifically, by examining expression level of Fabp7 gene or Fabp7 protein, it is possible to predict whether the onset and/or the progression of age-related hearing loss is/are advanced or delayed compared to normal, or to what degree it is/they are advanced or delayed compared to normal. In addition, the marker for noise-induced hearing loss according to the present invention is Fabp7 gene. That is, by examining expression level of Fabp7 gene or Fabp7 protein, it becomes possible to predict the degree of likelihood of noise-induced hearing loss.

The sample to be used to examine the expression level is not particularly limited, as long as Fabp7 gene or Fabp7 protein is expressed. It may be the cochlea, but blood or urine is preferable for non-invasive concerns.

### Examples

### (1) Expression of Fabp7 in Cochlea

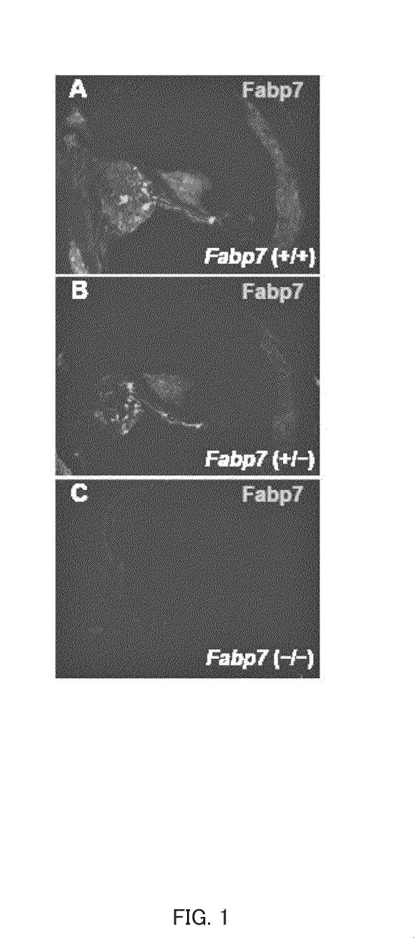

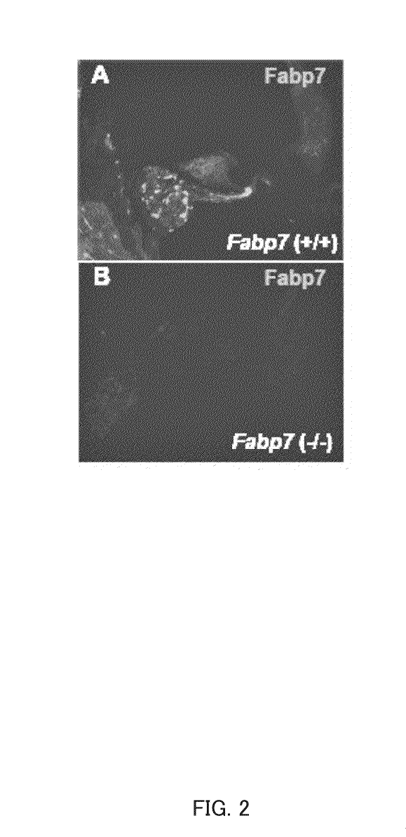

Fabp7 knockout mice (Fabp7 (−/−)), Fabp7 heterozygous knockout mice (Fabp7 (+/−)), and wild-type mice (Fabp7 (+/+))at 2 months (FIGS. 1) and 12 months (FIG. 2) of age were fixed by perfusion with 4% paraformaldehyde and their cochleae were dissected. The cochleae were fixed by immersion with the same fixative, decalcified in 10% EDTA, and embedded in O.T.C. Compound and their frozen sections were made. Immunostaining was then performed using an anti-Fabp7 antibody (Kurtz et al., Development, vol. 120, p. 2637-2649, 1994).

As previously reported, the expression of Fabp7 protein was detected in satellite cells in the spiral ganglion (SG), fibrocytes in the spiral limbus (SLim), supporting cells (inner phalangeal cells (IPC) and outer border cells of Hensen (OBCH)) in organ of Corti (OC).

FIGS. 1A, 1B, and 1C show the samples for which the conditions of staining and photographing are the same. As shown in FIG. 1, it was revealed that the expression of Fabp7 was reduced in the Fabp7 (+/−) mice to about a half level of that in the Fabp7 (+/+) mice and was absent in the Fabp7 (−/−) mice. In addition, as shown in FIG. 2, Fabp7 protein was expressed in the cochlea of the older Fabp7 (+/+) mice as in the younger mice but the expression was absent in Fabp7 (−/−) mice.

### (2) Survival of Fibrocytes and Neurons in Fabp7 Knockout Mice

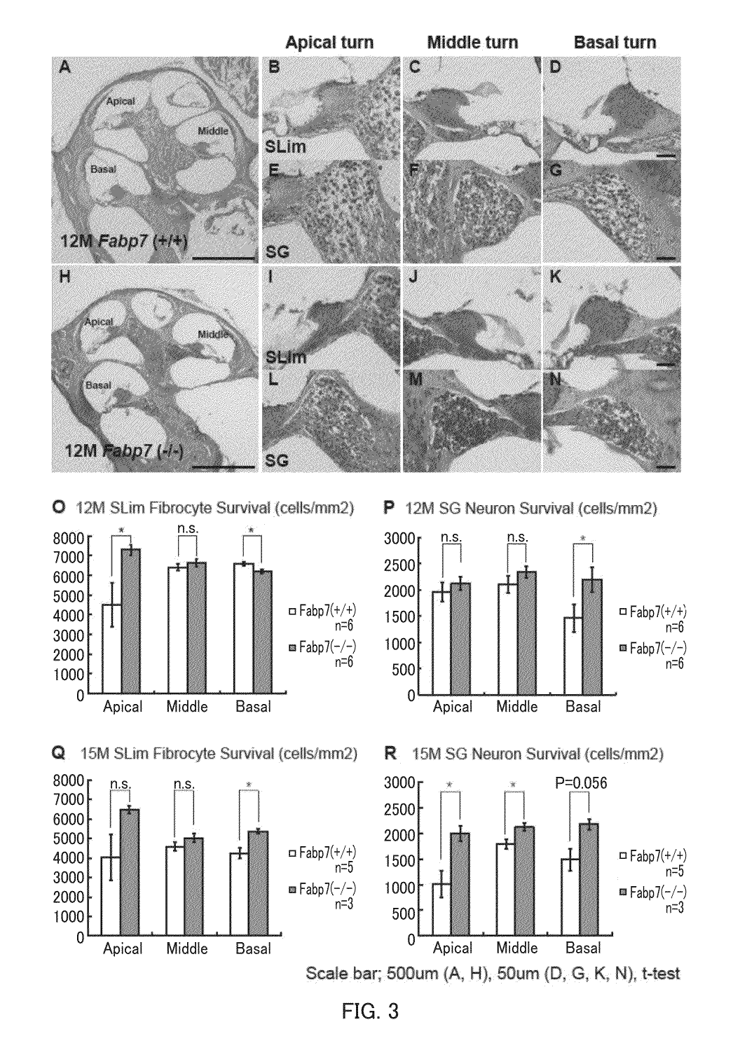

Fabp7 knockout mice (Fabp7 (−/−)) and wild-type mice (Fabp7 (+/+)) at 12 and 15 months of age were fixed by perfusion with 4% paraformaldehyde (12-month old) or Bouin's solution (15-month old) and their cochleae were dissected. The cochleae were fixed by immersion with the same fixative, decalcified in 10% EDTA, and embedded in O.T.C. Compound and their frozen sections were made. HE staining was then performed and morphology of the inner ear was assessed. FIGS. 3A to 3N show magnified images of the entire cochlea as well as apical, middle, and basal turns of mice at 12 months of age.

In the cochleae of the Fabp7 (+/+) mice at 12 months of age (FIGS. 3A to 3G), fibrocytes in the spiral limbus (SLim) and the spiral ganglion (SG) neurons were found to be lost whereas loss was slight in the cochleae of the Fabp7 (−/−) mice.

Next, fibrocytes in the spiral limbus (SLim) and spiral ganglion (SG) neurons were counted (FIGS. 1O to 1R). Cells were counted in three sections per animal for each turn and the averages of the cell numbers in the three sections were calculated as the number of cells per unit area (1 mm²). In the spiral limbus, a significantly larger number of fibrocytes survived in the Fabp7 (−/−) mice than in the Fabp7 (+/+) mice for the apical turns of the 12-month-old mice and the basal turns of the 15-month-old mice. In the spiral ganglion, a significantly larger number of neurons were survived in the Fabp7 (−/−) mice than in the Fabp7 (+/+) mice for the basal turns of 12-month-old mice and the apical and middle turns of the 15-month-old mice. As apparent from the above, in the Fabp7 (−/−) mice, age-related degeneration of the cochlea was mild, and loss of neurons and fibrocytes was suppressed.

### (3) Survival of Hair Cells in Fabp7 Knockout Mice

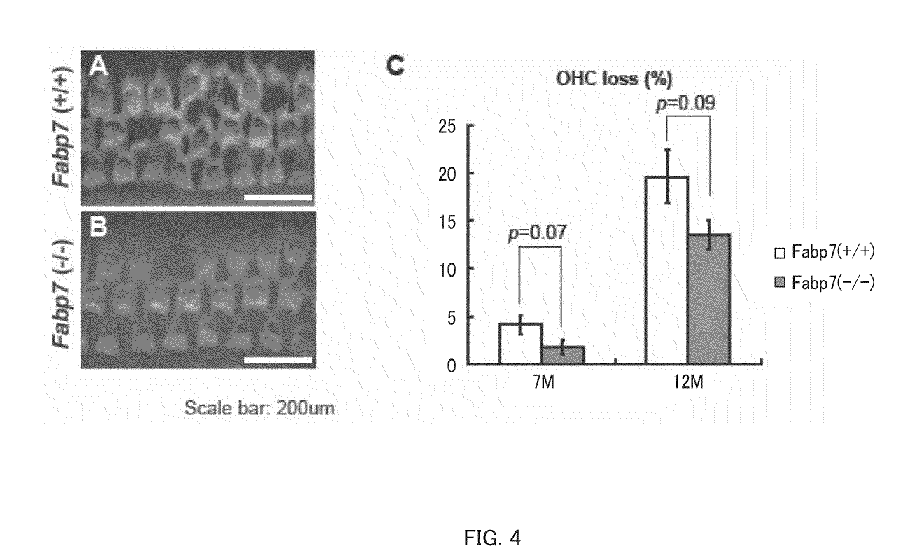

Fabp7 knockout mice (Fabp7 (−/−)) and wild-type mice (Fabp7 (+/+)) at 7 and 12 months of age were fixed by perfusion with 4% paraformaldehyde and their cochleae were dissected. The cochleae were fixed by immersion with the same fixative and decalcified in 10% EDTA. Hair cells were then stained with phalloidin-rhodamine conjugate. The organs of Corti were dissected and mounted on glass slides (surface preparation method). The number of outer hair cells in the apical region was measured, and percentage of missing outer hair cells was determined relative to an expected number of outer hair cells (Note: three outer hair cells are expected to be present for one inner hair cell). Three or more fields were observed, each of which contained three rows of ten outer hair cells and averages of the cell numbers in the fields were calculated. FIG. 4A shows morphology of outer hair cells of a Fabp7 (+/+) mouse at 12 months of age and FIG. 4B shows morphology of outer hair cells of a Fabp7 (−/−) mouse at 12 months of age. FIG. 4C shows percentage of missing outer hair cells (OHC loss).

It was confirmed that morphological integrity of the outer hair cells were lost in the Fabp7 (+/+) mice (FIG. 4A) due to loss of cells by randomly occurring cell death whereas the outer hair cells were well aligned in the Fabp7 (−/−) mice (FIG. 4B). In addition, as shown in FIG. 4C, from quantitative considerations, the OHC loss was lower in the Fabp7 (−/−) mice than in the Fabp7 (+/+) mice both at 7 and 12 months of age. As apparent from the above, age-related loss of hair cells was suppressed in the Fabp7 (−/−) mice.

(4) Delay of Onset and/or Progression of Age-Related Hearing Loss in Fabp7 Knockout Mice

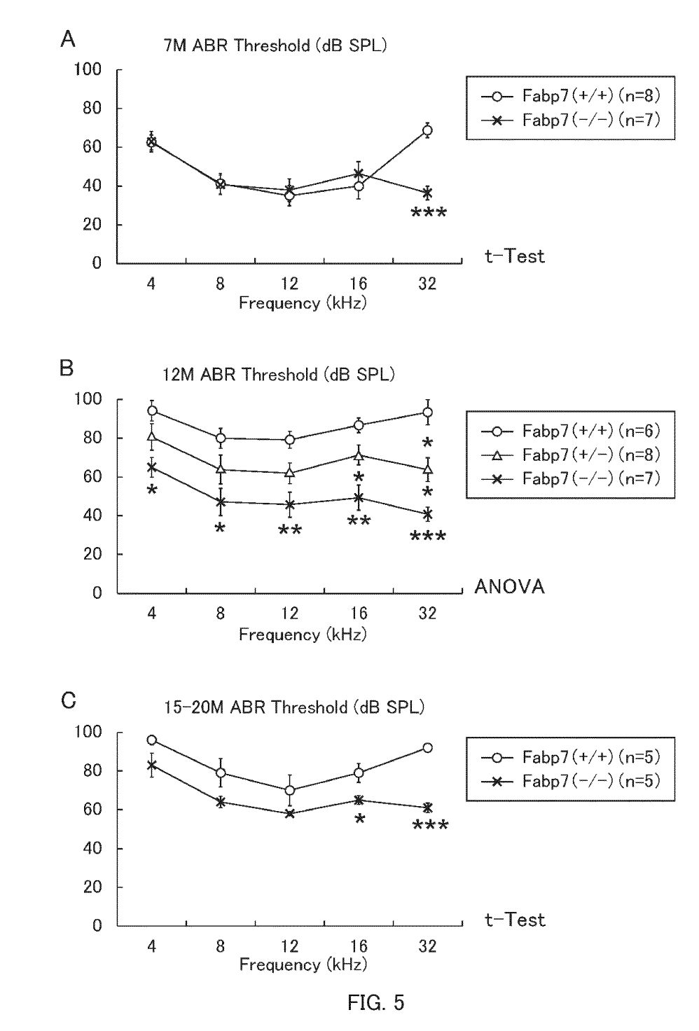

Hearing thresholds were measured by auditory brain-stem response (ABR) in Fabp7 knockout (Fabp7 (−/−)) mice, Fabp7 heterozygous knockout (Fabp7 (+/−)) mice and wild-type (Fabp7 (+/+)) mice at 7, 12, and 15-20 months of age (FIGS. 5A to 5C). The mice were anesthetized with xylazine and ketamine and then hearing thresholds were measured using tone burst stimuli at five different frequencies of 4, 8, 12, 16, and 32 kHz.

As shown in FIG. 5A, the Fabp7 (+/+) mice at 7 months of age showed increases in hearing threshold at high frequency of 32 kHz; whereas the Fabp7 (−/−) mice of the same age showed no increase in hearing threshold. As shown in FIG. 5B, the Fabp7 (+/+) mice at 12 months of age showed increases in hearing threshold at all frequencies; the Fabp7 (−/−) mice showed no increase in hearing threshold; and the Fabp7 (+/−) mice showed increases to a middle level between that of the wild-type animals and that of the homozygous knockout animals. As shown in FIG. 5C, the Fabp7 (−/−) mice at 15-20 months of age had lower hearing thresholds than the Fabp7 (+/+) mice over the entire acoustic range and, in particular, the thresholds were significantly low at 16 and 32 kHz.

From the results obtained with the animals at 12 months of age (FIG. 5B) as well as the result that the expression of Fabp7 protein was reduced by about half in the Fabp7 (+/−) mice (FIGS. 1A to 1C), it is revealed that the hearing is impaired in proportion to the number of wild-type Fabp7 loci. Accordingly, Fabp7 protein can be a quantitative marker indicating onset and/or level of progression of age-related hearing loss.

### (5) Reduction of Temporary Elevation of Hearing Threshold in Fabp7 Knockout Mice

In this example, Fabp7 knockout (Fabp7 (−/−)) mice and wild-type (Fabp7 (+/+)) mice were exposed to noise at 2 months of age (9-12 weeks old) and their hearing thresholds were then measured using auditory brain-stem response (ABR) to measure temporary threshold shift (TTS). Specific details are as follows.

For acoustic exposure, unanesthetized mice were housed in a wire-mesh cage placed in a soundproof booth (custom-ordered, KAWAI). The mice were subjected to excessive acoustic noise produced by a combination of a noise generator (SF-06, RION), a frequency filter (3611, NF), a power amplifier (D-75A, Crown), and a speaker (2446H, JBL) (A-D) under conditions of 8-10 kHz of the band noise, 89 dB SPL of the sound pressure level, and 2 hours-period.

Mice were anesthetized with xylazine and ketamine before and after exposure to noise and ABR was then measured using tone burst stimuli at five different frequencies of 4, 8, 12, 16, and 32 kHz. In order to examine temporary threshold shift (TTS), ABR was measured before the acoustic exposure (B), 4 hours after the exposure, and 7 days after the exposure and shift in hearing threshold before and after the exposure were calculated (C, D).

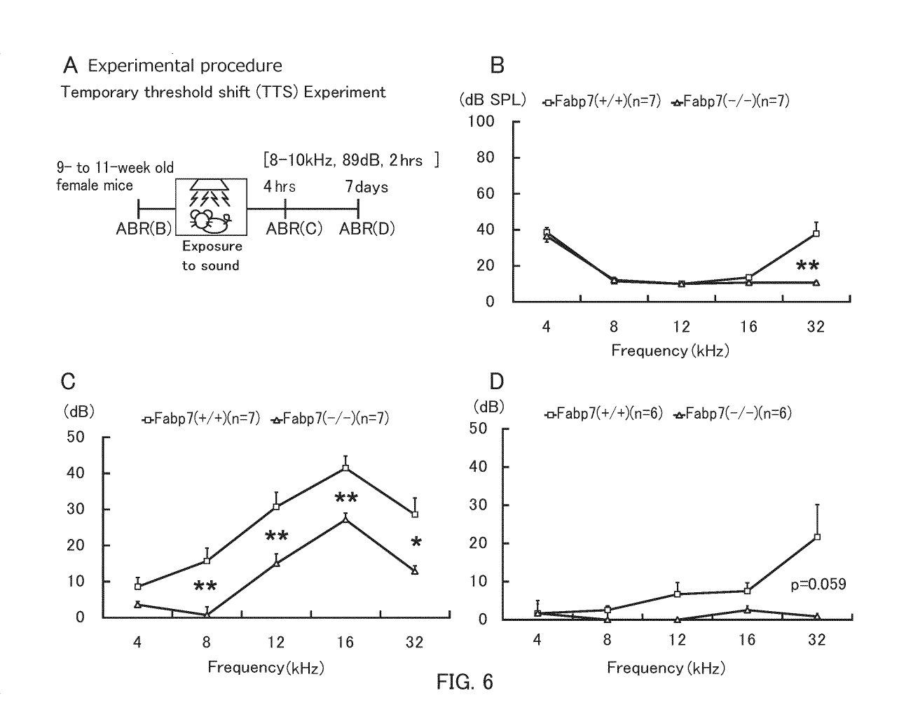

As shown in FIG. 6B, before the acoustic exposure, the Fabp7 (+/+) mice at 2 months of age (9-12 week old) already showed increase in hearing threshold at 32 kHz whereas the Fabp7 (−/−) mice showed no increase in the hearing threshold. No differences in hearing threshold was observed between them at the other frequencies.

At four hours after the exposure, the hearing threshold of Fabp7 (+/+) mice was elevated over the entire acoustic range whereas the elevation of the hearing threshold at 8, 12, 16, and 32 kHz was significantly reduced in the Fabp7 (−/−) mice (FIG. 6C). On seven days after the exposure, the Fabp7 (−/−) mice showed improvement of the hearing threshold over the entire acoustic range to a level similar to that before the exposure whereas the hearing threshold tended to be poorly improved at 32 kHz in the Fabp7 (+/+) mice (FIG. 6D).

As apparent from the above, Fabp7 deficiency results in reduction of temporary threshold elevation due to acoustic exposure.

### INDUSTRIAL APPLICABILITY

In accordance with the present invention, animal models resistant to hearing loss can be provided.

1. An animal model resistant to hearing loss, wherein expression of a fatty acid binding protein 7 protein is suppressed in the animal.

2. The animal model according to claim 1, wherein the hearing loss is age-related hearing loss, and wherein onset or progression of the age-related hearing loss is delayed.

3. The animal model according to claim 1, wherein the hearing loss is noise-induced hearing loss, and wherein the noise-induced hearing loss being reduced.

4. A method, comprising of measuring hearing with the passage of age in the animal model of claim 2.

5. A method, comprising of measuring hearing after acoustic exposure in the animal model of claim 3.

6. A method of screening for a gene involved in age-related hearing loss, comprising searching for a gene showing expression in the animal model of claim 2 in a manner different from that of the gene in an animal exhibiting normal age-related hearing loss.

7. A method of screening for a gene involved in noise-induced hearing loss, comprising searching for a gene showing expression in the animal model of claim 3 in a manner different from that of the gene in an animal exhibiting normal noise-induced hearing loss.

8. (canceled)

9. A method of screening for a substance as a marker for age-related hearing loss, comprising searching for a compound present in the animal model of claim 2 in an amount or a concentration different from that of the compound in an animal exhibiting normal age-related hearing loss.

10. A method of screening for a substance as a marker for noise-induced hearing loss, comprising searching for a compound present in the animal model of claim 3 in an amount or a concentration different from that of the compound in an animal exhibiting normal noise-induced hearing loss.

11. A method of producing an animal model with delayed onset and/or progression of age-related hearing loss, comprising suppressing expression of fatty acid binding protein 7 gene in an animal.

12. A method of producing an animal model with reduced noise-induced hearing loss, comprising suppressing expression of fatty acid binding protein 7 gene in an animal.

13. A method of delaying onset or progression of age-related hearing loss in a vertebrate, comprising suppressing expression of fatty acid binding protein 7 gene in the vertebrate.

14. A method of reducing noise-induced hearing loss in a vertebrate, comprising suppressing expression of fatty acid binding protein 7 gene in the vertebrate.

15. A method of screening for a compound capable of delaying onset or progression of age-related hearing loss, comprising examining whether a candidate compound inhibits expression of fatty acid binding protein 7.

16. A method of screening for a compound capable of reducing noise-induced hearing loss, comprising examining whether a candidate compound inhibits expression of fatty acid binding protein 7.

17. A marker for age-related hearing loss, the marker being a fatty acid binding protein 7 gene.

18. A marker for noise-induced hearing loss, the marker being a fatty acid binding protein 7 gene.
